(19) United States
(12) Patent Application Publication
(10) Pub. No.: US 2019/0374152 A1
(43) Pub. Date: Dec. 12, 2019

(54) PATIENT MONITORING SYSTEM

(71) Applicants: George Wayne, Miami, FL (US); Mark Jacobs, Miami Beach, FL (US); Carlos Eduardo Lovera, Southwest Ranches, FL (US); Anthony Perez-Sanz, Miami, FL (US); Aaron Bronshtein, Oakland, NJ (US)

(72) Inventors: George Wayne, Miami, FL (US); Mark Jacobs, Miami Beach, FL (US); Carlos Eduardo Lovera, Southwest Ranches, FL (US); Anthony Perez-Sanz, Miami, FL (US); Aaron Bronshtein, Oakland, NJ (US)

(21) Appl. No.: 16/547,529

(22) Filed: Aug. 21, 2019

Related U.S. Application Data
(63) Continuation-in-part of application No. PCT/US18/19066, filed on Feb. 21, 2018.

(51) Int. Cl. A61B 5/20; A61B 5/00

(52) U.S. Cl. CPC A61B 5/208; A61J 1/10; A61B 5/204; A61B 5/742

(57) ABSTRACT

A patient monitoring system may include a first-fluid-housing, and a drain in communication with the first-fluid-housing that pulls a first non-serosanguinous fluid from a patient into the first-fluid-housing. The system may also include a sensor that measures the first non-serosanguinous fluid entering the first-fluid-housing. The system may further include a computer-controller in communication with the sensor and drain, the computer-controller includes a profile that identifies the patient, the first-fluid-housing, and records the sensor's first non-serosanguinous fluid measurements that any user can retrieve based upon permissions granted to each user.

### BACKGROUND

The embodiments relate to the field of patient monitoring systems.

There are many different types of patient monitoring systems. One type of patient monitoring system involves checking a patient's fluid output.

### SUMMARY

According to an embodiment, a patient monitoring system may include a first-fluid-housing, and a drain in communication with the first-fluid-housing that pulls a first-fluid from a patient into the first-fluid-housing. The system may also include a sensor that measures the first-fluid entering the first-fluid-housing. The system may further include a computer-controller in communication with the sensor and drain, the computer-controller includes a profile that identifies the patient, the first-fluid-housing, and records the sensor's first-fluid measurements that any user can retrieve based upon permissions granted to each user.

The system may additionally include a second-fluid-housing in communication with the drain that pulls a second-fluid generated by the patient into the second-fluid-housing, and the profile identifies the patient, the second-fluid-housing, and records the sensor's second-fluid measurements that any user can retrieve based upon permissions granted to each user. The system may also include an interface connecting the computer-controller to any user's computer based upon permissions granted to each user.

The sensor may record in the profile images of the first-fluid generated by the patient that any user can retrieve based upon permissions granted to each user so the user can interpret them and make clinical decisions remotely. The user may be human and/or machine-analysis, and images' characteristics include color, consistency, homogeneity, particulates, flow, volume, consistency, weight, composition, temperature, and/or clinically correlated diagnostics.

The sensor may measure the first-fluid entering the first-fluid-housing continuously, intermittently, in aggregate after the first-fluid-housing is exchanged, by flowrate, and by timestamp. The users and the patient communicate with each other based upon their permissions, and this communication allows the patient to directly enter descriptions of their subjective clinical experience into the profile. The computer-controller includes customizable thresholds that are set by the user based upon their permissions and prevents inappropriate settings of the drain, sensor, and/or user permissions.

### BRIEF DESCRIPTION OF THE DRAWINGS

FIG. 1 is a schematic block diagram of a system in accordance with the embodiments.

FIG. 2 are three fluid housing of the system of FIG. 1.

FIG. 3 is a view of the components of the system of FIG. 1.

FIG. 4 is illustrates how the system of FIG. 1 is connected to a patient.

FIG. 5 shows how a patient is paired with the system of FIG. 1.

FIG. 6 illustrates basic settings that a are programmed at each initialization for a patient using the system of FIG. 1.

FIG. 7 illustrates how a nurse can leave annotations for future reference in the system of FIG. 1.

FIG. 8 illustrates information flows within the system of FIG. 1.

FIGS. 9 and 10 illustrate how patients can download the Patient App to their mobile devices and begin interacting with the system of FIG. 1.

FIGS. 11, 12, and 13 illustrate clinical and administrative/research users accessing their user profiles within the system of FIG. 1.

FIG. 14 illustrates thresholds to inappropriate settings within the system of FIG. 1.

FIG. 15 illustrates thresholds to inappropriate settings within the system of FIG. 1.

FIG. 16 illustrates changing a fluid housing within the system of FIG. 1.

FIG. 17 illustrates patient-controlled charting within the system of FIG. 1.

FIG. 18 illustrates physician parameters for fluid outputs within the system of FIG. 1.

FIG. 19 illustrates fluid color within the system of FIG. 1.

FIG. 20 is an exemplary configurations of the system of FIG. 1.

FIG. 21 is another exemplary configurations of the system of FIG. 1.

FIGS. 22-24 are exemplary images captured by the system of FIG. 1.

FIG. 25 are exemplary configurations of the system of FIG. 1.

FIG. 26 are exemplary configurations of the system of FIG. 1.

### DETAILED DESCRIPTION

Embodiments will now be described more fully hereinafter with reference to the accompanying drawings, in which preferred embodiments are shown. Like numbers refer to like elements throughout.

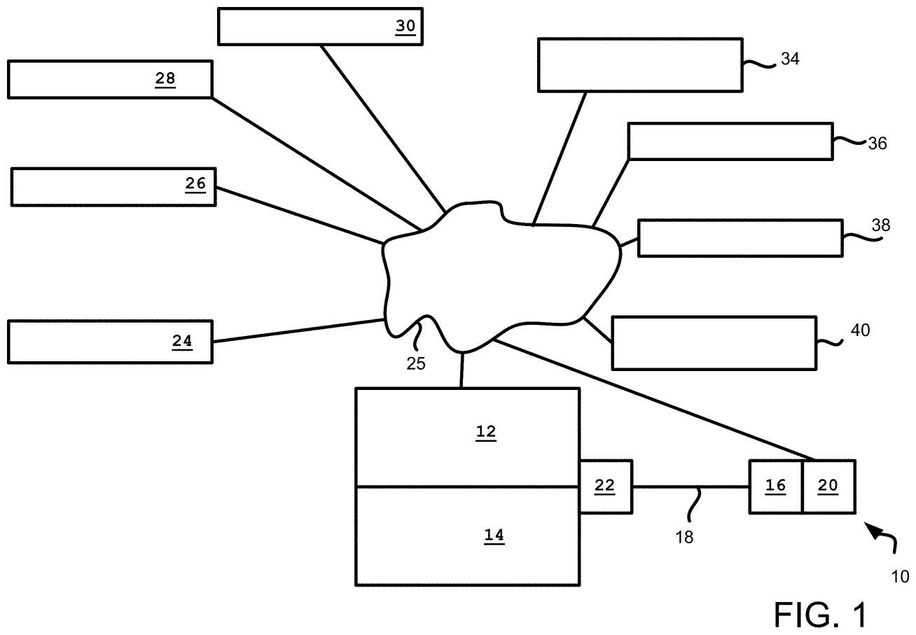

With reference now to FIGS. 1-3, a patient monitoring system 10, e.g. Flolink, is initially described. In one embodiment, the system 10 includes a first-fluid-housing 12. In another embodiment, the system 10 includes multiple fluid-housing such as second-fluid-housing 14. The system also includes a drain 16 in communication with the first-fluid-housing 12 that pulls a first-fluid 18 from a patient 20 into the first-fluid-housing. In one embodiment, the drain 16 is an active drain such as a vacuum pump, wall suction, Jackson-Pratt (JP), and Hemovac. In another embodiment, the drain 16 a passive drain, e.g. T-tube, Penrose, that rely on gravity to evacuate first-fluid 18 from the patient 20.

The system 10 also includes a sensor 22 that measures the first-fluid 18 entering the first-fluid-housing 12. The sensor 22 measures properties of the first-fluid 18 such as temperature, color, weight, flow rate, total flow, consistency, homogeneity, particulates, flow, volume, consistency, composition, and clinically correlated diagnostics. The system 10 can include multiple sensors 22 that measure the different properties of the first-fluid 18.

The system 10 further includes a computer-controller 24 in communication with the sensor 22 and drain 16, the computer-controller includes a profile 26 that identifies the patient 20, the first-fluid-housing 12, and records the sensor's 22 first-fluid 18 measurements that any user 28 can retrieve based upon permissions 30 granted to each user. In one embodiment, the computer-controller 24 includes a processor and is an electronic device such as a tablet computer, desktop computer, laptop computer, cellphone, and/or application-specific integrated circuit. In another embodiment user 28 are professionals caring for the patient 20 such as doctors, nurses, clinicians, specialists, and insurance adjusters.

In one embodiment, the system 10 additionally includes a second-fluid-housing 14 in communication with the drain 16 that pulls a second-fluid generated by the patient 20 into the second-fluid-housing, and the profile 26 identifies the patient, the second-fluid-housing, and records the sensor's second-fluid measurements that any user 28 can retrieve based upon permissions granted to each user. In another embodiment, the system 10 also includes an interface 34 connecting the computer-controller 24 to any user's 28 computer 36 based upon permissions granted to each user.

In one embodiment, the sensor 22 records in the profile 26 images 38 of the first-fluid 18 generated by the patient 20 that any user 28 can retrieve based upon permissions granted to each user so the user can interpret them and make clinical decisions remotely. In another embodiment, the user 28 is human and/or includes machine-analysis, and images' 38 characteristics include color, consistency, homogeneity, particulates, flow, volume, consistency, weight, composition, temperature, fluid pH, presence of pathogens, and/or clinically correlated diagnostics.

In one embodiment, the sensor 22 measures the first-fluid 18 entering the first-fluid-housing 12 continuously, intermittently, in aggregate after the first-fluid-housing is exchanged, by flowrate, and by timestamp. In another embodiment, the users 28 and the patient 20 communicate with each other based upon their permissions, and this communication allows the patient to directly enter descriptions of their subjective clinical experience into the profile 26. In another embodiment, the computer-controller 24 includes customizable thresholds that are set by the user 28 based upon their permissions and prevents inappropriate settings of the drain 16, sensor 22, and/or user permissions.

In one embodiment, the patient monitoring system 10 includes a first-fluid-housing 12, a drain 16 in communication with the first-fluid-housing that pulls a first-fluid 18 from a patient 20 into the first-fluid-housing. The system 10 also includes a sensor 22 that measures the first-fluid 18 entering the first-fluid-housing 12. The system further includes a computer-controller 24 in communication with the sensor 22 and drain 16, the computer-controller includes a profile 26 that identifies the patient 20, the first-fluid-housing 12, and records the sensor's 22 first-fluid 18 measurements that any user 28 can retrieve based upon permissions granted to each user. The system additionally includes a second-fluid-housing 14 in communication with the drain 16 that pulls a second-fluid generated by the patient 20 into the second-fluid-housing, and the profile 26 identifies the patient, the second-fluid-housing, and records the sensor's 22 second-fluid measurements that any user 28 can retrieve based upon permissions granted to each user. The system also includes an interface 34 connecting the computer-controller 24 to any user's 28 computer based upon permissions granted to each user.

In one embodiment, the patient monitoring system 10 includes a first-fluid-housing 12, a drain 16 in communication with the first-fluid-housing that pulls a first-fluid 18 from a patient 20 into the first-fluid-housing. The system 10 also includes a sensor 22 that measures the first-fluid 18 entering the first-fluid-housing 12. The system 10 further includes a computer-controller 24 in communication with the sensor 22 and drain 16, the computer-controller includes a profile 26 that identifies the patient 20, the first-fluid-housing 12, and records the sensor's 22 first-fluid 18 measurements that any user 28 can retrieve based upon permissions granted to each user, the sensor records in the profile images of the first-fluid generated by the patient that any user can retrieve based upon permissions granted to each user so the user can interpret them and make clinical decisions remotely, and the users and the patient communicate with each other based upon their permissions, and this communication allows the patient to directly enter descriptions of their subjective clinical experience into the profile.

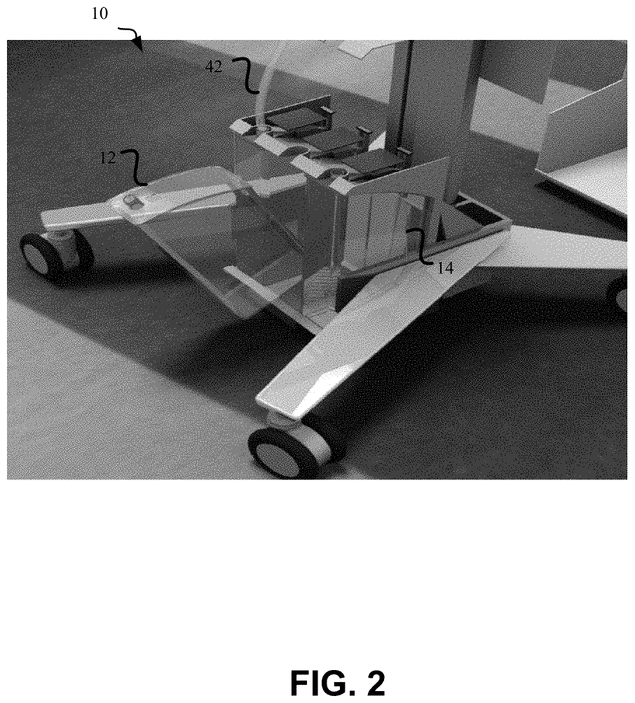

With respect to FIG. 2, a nurse brings system 10, e.g. FloLink, into the patient's room. The nurse inserts the fluid housing 18 into their docks (locks them flush against the sensors 22) and connects all the tubing 42 (as labeled on tubing set) to the fluid receptacles.

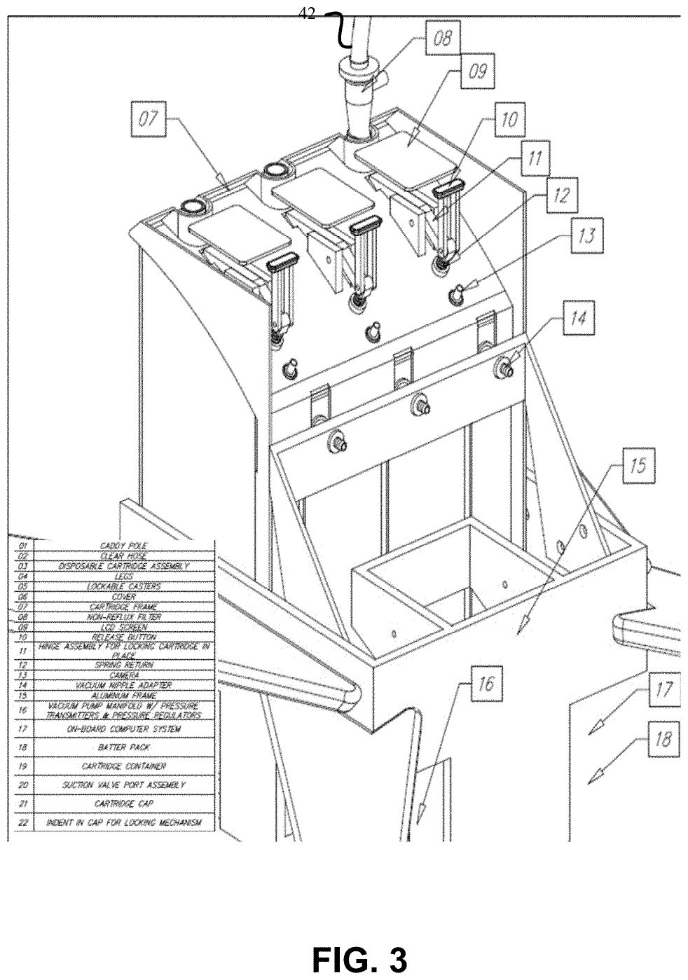

With respect to FIG. 3, a hinged locking system (11) presses the fluid receptacle against its associated capacitive sensor (24) and a vacuum nipple (14) which interfaces with a needleless female end port on the receptacle reinforcing this positioning. The view on the left shows FloLink with the back opened, demonstrating the space where the circuitry and microcontroller are stored.

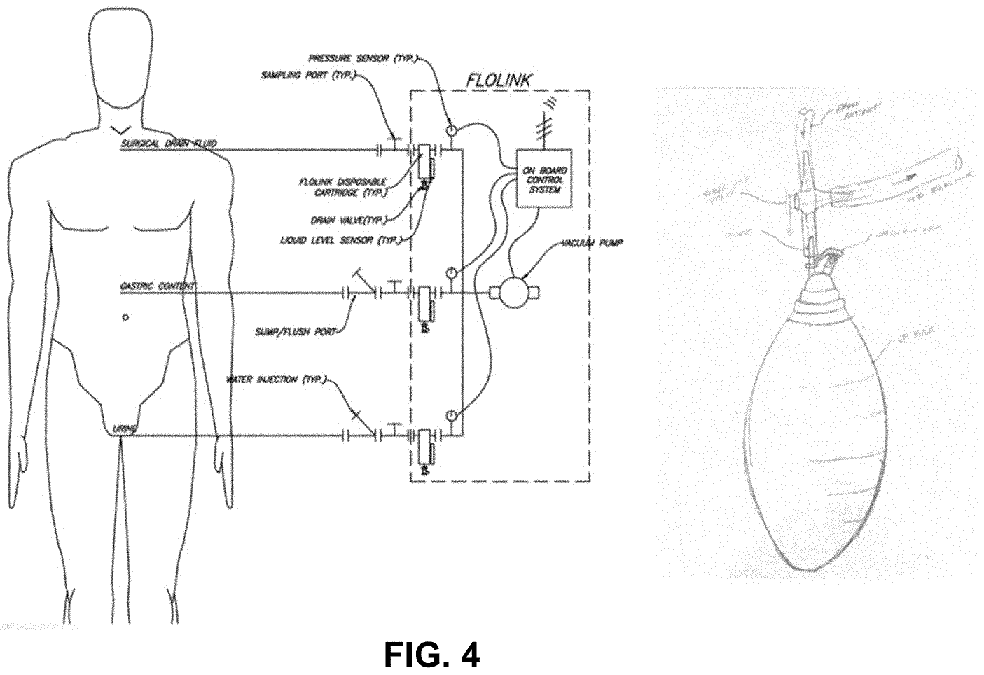

With respect to FIG. 4, all tubing 42 is connected (as labeled on the tubing set) to the fluid housing 18, with the patient end connected to a Y-connector bypassing the standard drain receptacle unit.

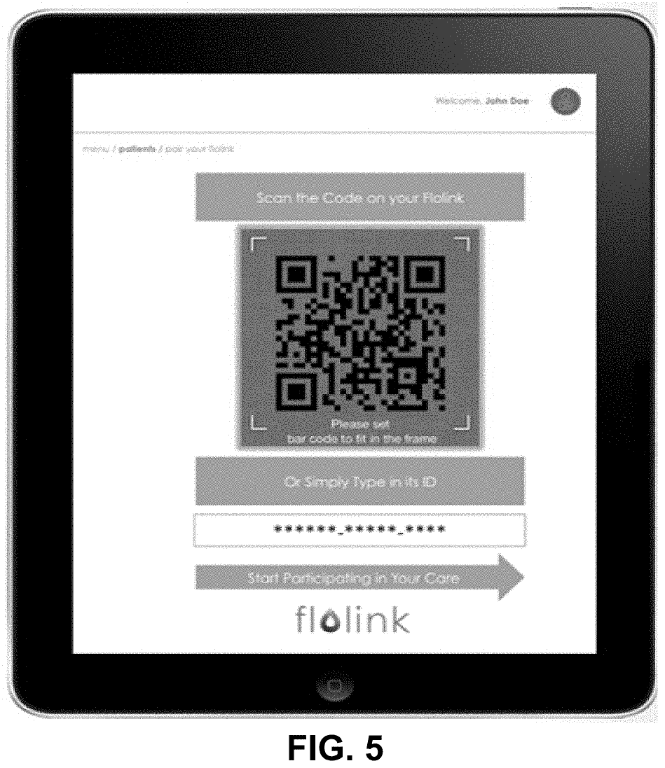

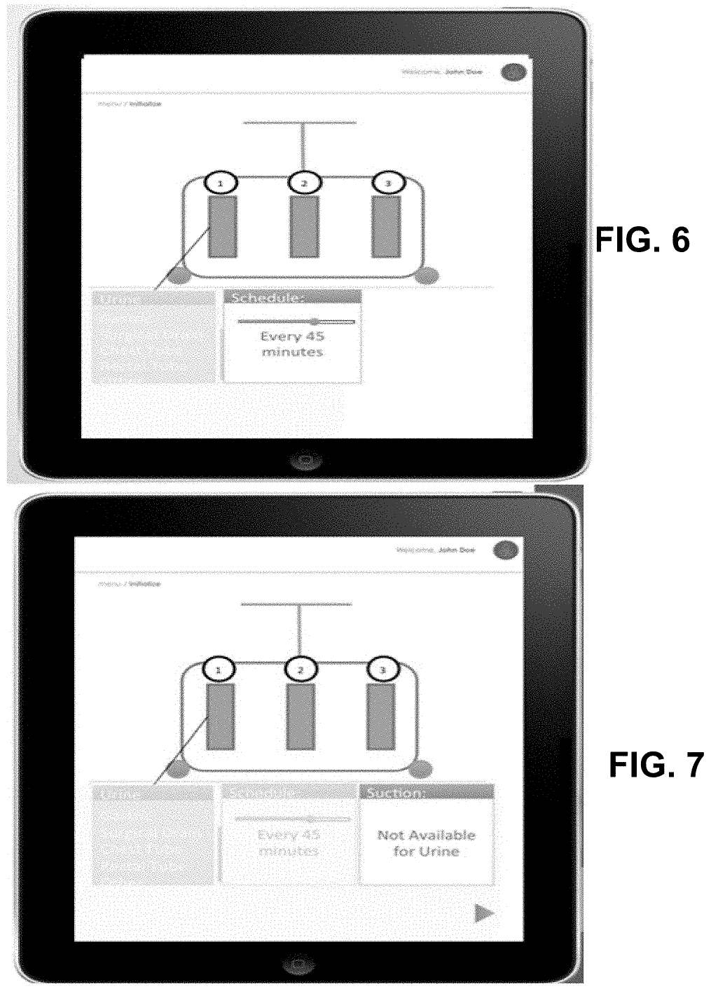

With respect to FIG. 5, the nurse 28 then uses the on-board tablet/smartphone, following software-guided steps, to pair system 10 with the patient's identity and room number. Each FloLink unit has a built-in virtually generated barcode for this purpose.

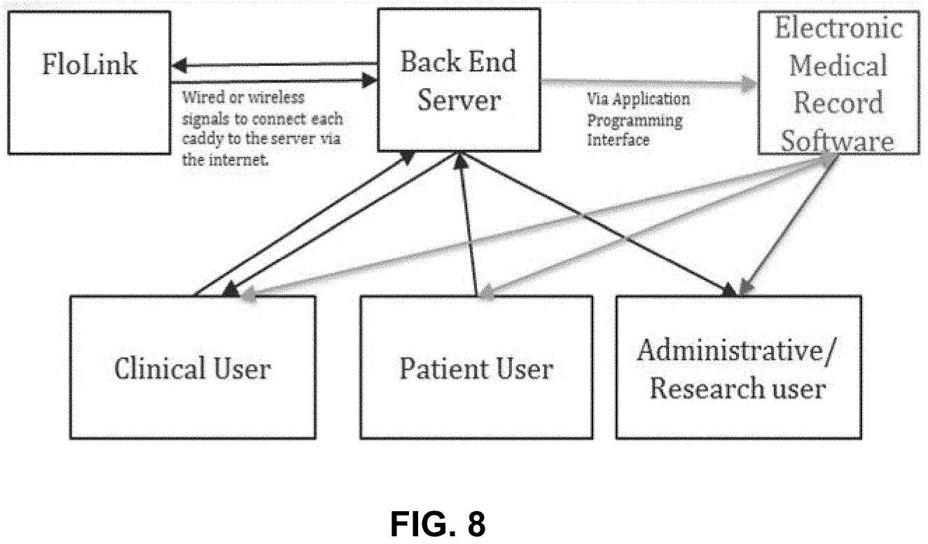

With respect to FIG. 8, system 10 begins collecting information as scheduled by the healthcare professional. Information gathered by FloLink is sent to a back-end server, which is accessed by patients and clinicians, with different user profiles. Two-way communication allows data collected by FloLink to transfer to the server, and instructions, settings, and annotations to transfer back to FloLink. Each user-type can access their profile via desktop or mobile app. Alternatively, information may transfer via the hospital Electronic Medical Record between each mentioned user.

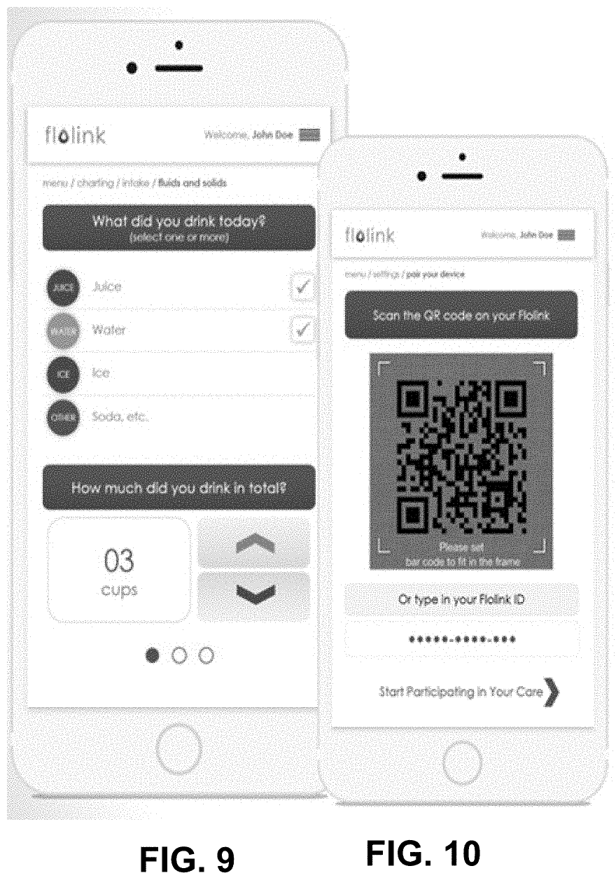

With respect to FIGS. 9 and 10, once the system 10 is initialized, patients 20 can download the FloLink Patient App to their mobile devices and begin interacting with the FloLink software and the EMR by entering responses to prompts requesting information about: Clinical Review of Systems (how patients feel), e.g., Nausea, vomiting, diarrhea, Bowel and Bladder Habits/changes, Food Consumption, and Fluid Consumption.

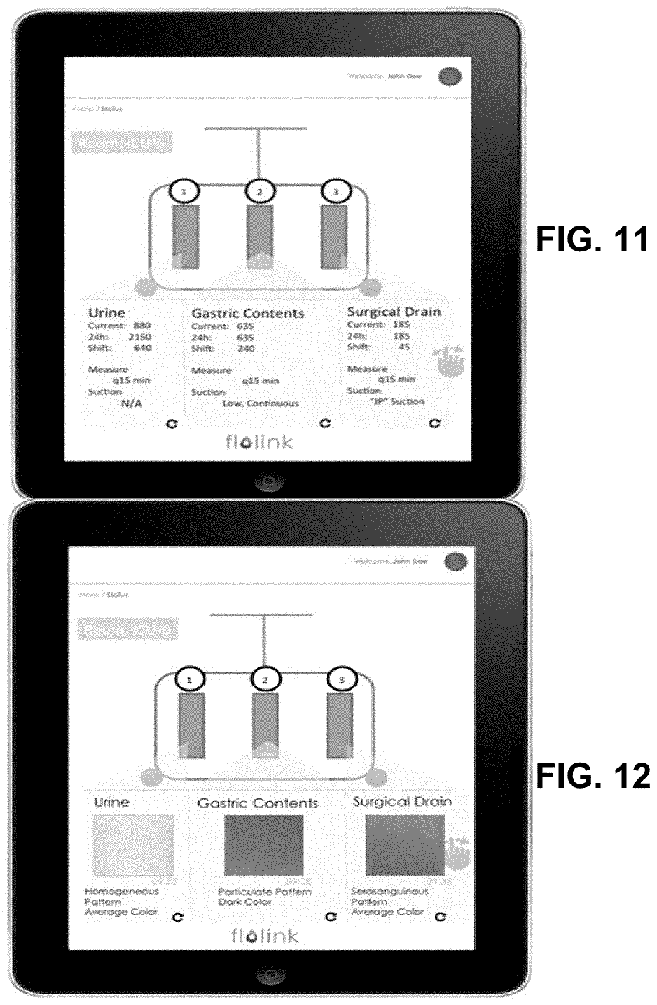

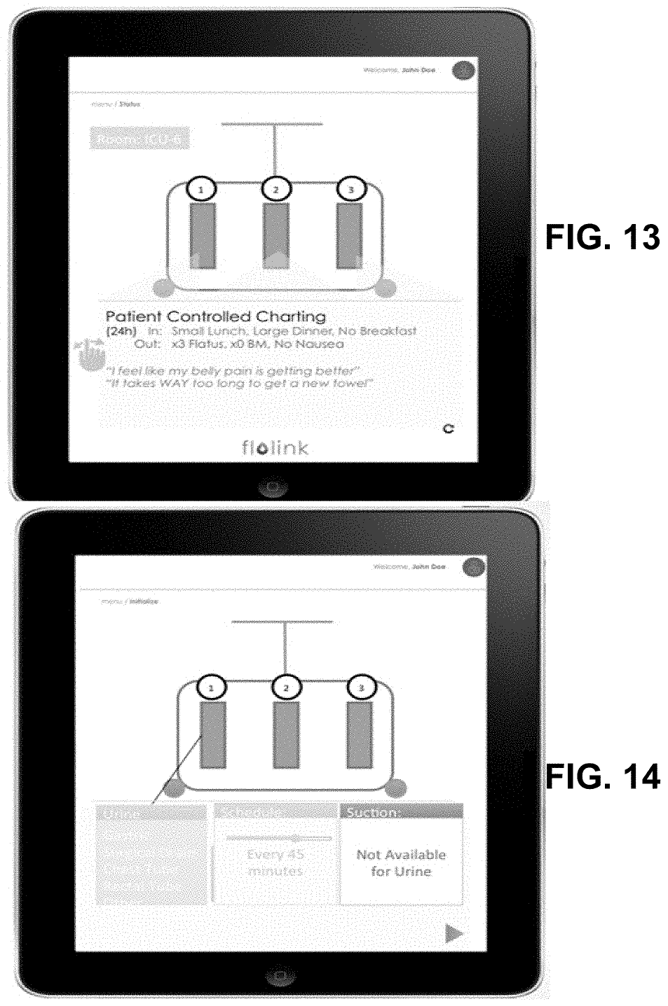

In addition, clinical and administrative/research users can access their user profiles, from which they can change preferences for updating schedules and add/remove patients and associated FloLinks to their working directories. The on-board FloLink screen displays a status board for each patient, providing local communication of data that can be accessed by clinical and research users via mobile or desktop applications. FIGS. 11-13 provide representative screens of this kind of information.

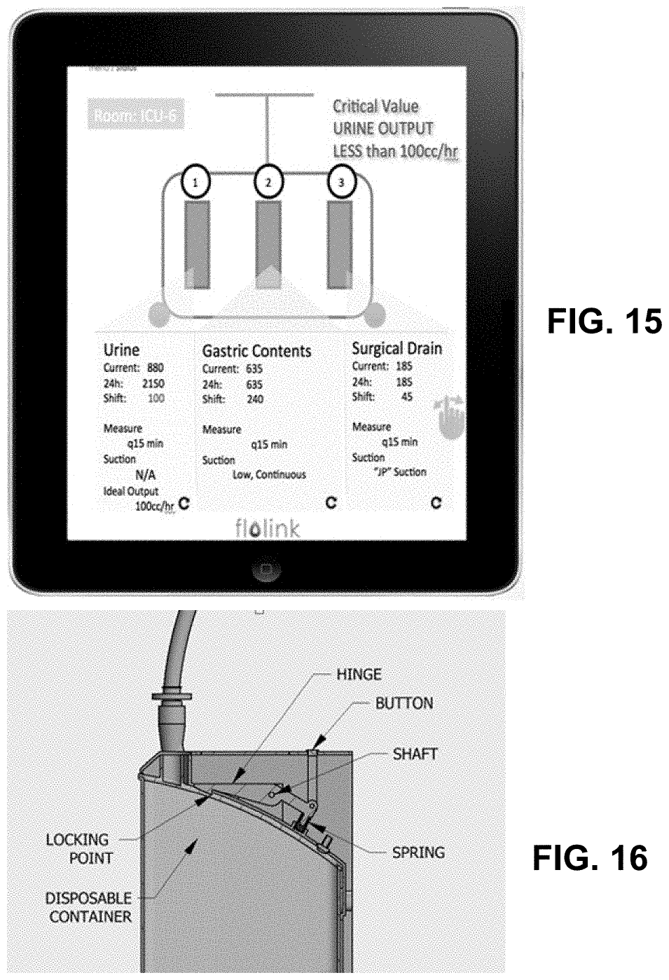

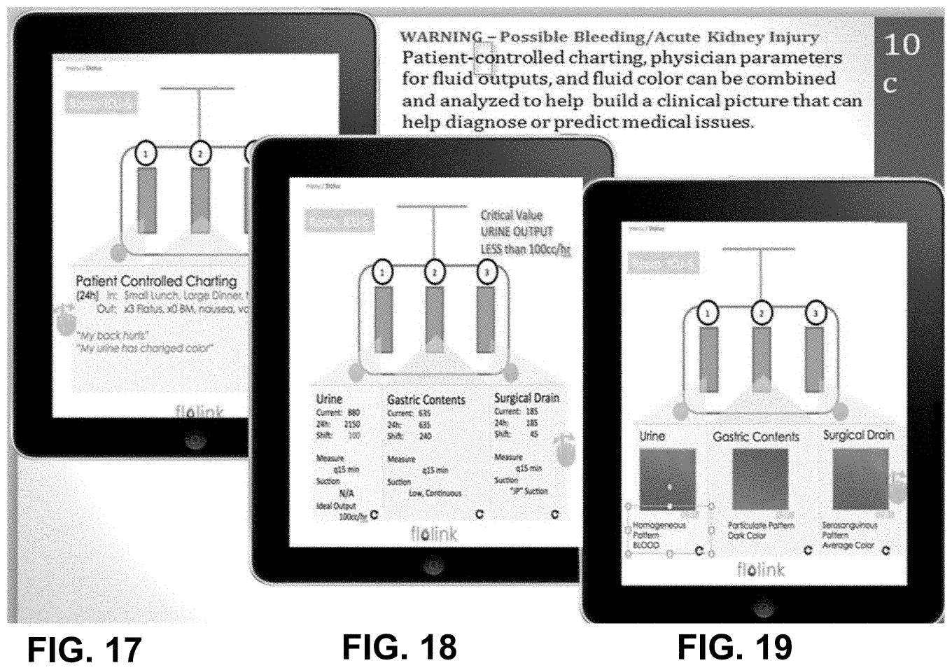

With respect to FIG. 15, settings can be customized to different parameters to alert clinicians and nurses to concerning changes in patient's fluid outputs. These can be predictors of worrisome changes in a patient's condition.

With respect to FIG. 16, when the nurse or technician needs to change a FloLink fluid receptacle, she presses the release button on the top of FloLink (labeled above), which ejects the fluid receptacle and sends an electronic signal to the system to save the most recent volumetric measurements taken from the old receptacle and add to them measurements from the new receptacle to provide an accurate representation of cumulative volume in a given period of time. The software also allows this user to dissociate a patient from his FloLink (i.e. during discharge or discontinuation of fluid monitoring), or to annotate when external fluid has been added to the system—e.g. during irrigation.

In one embodiment, the system 10 includes a fluid housing 12, a set of sensors 22 in direct or indirect communication with fluids 18 generated by the patient 20, a controller 24 in communication with the sensors, a profile 26 in the controller that identifies the fluid housing and records output from the set of sensors, a server in communication with the profile, a digital application in communication with the server, a set of computer programs which organize communication between the set of sensors, the controller, the profile, the server, pre-existing electronic medical records, and the digital application and which provide patient identification and recorded data from the set of sensors to a user based upon permissions.

In another embodiment, the system 10 includes a set of computer programs enables users 28 to input and delete data as well as control-parameters of at least one of the set of sensors 22 and controller 24. The server, users, and the patient communicate with each other based upon their permissions. This communication allows the patient 20 to directly enter descriptions of their subjective clinical experience into the electronic medical record (EMR). For example, the patient 20 communicating directly with the medical record by way of a digital application that s/he experienced a bout of diarrhea. Clinical information like this is quickly transferred to the record without requiring interrogation by multiple layers of clinicians.

In one embodiment, the fluid 18 includes urine, enteric contents, surgically placed drains, thoracic drains, fluids from negative pressure wound therapy, and fluids dispensed by the patient into the fluid housing. In another embodiment, the digital application facilitates communication by providing graphical user interfaces 34 to users 28 based upon their permissions.

In one embodiment, each fluid 18 is graphically presented on the digital application 34. The output data from the set of sensors 22 is graphically presented on the digital application 34 and which is stored on the server, e.g. computer-controller 24.

In one embodiment, the set of sensors 22 captures images 38 (moving and static, photographic and infrared), and video of patient 20 fluids 18 and data about their flow, volume, consistency, and weight. The set of computer programs can use data acquired by the set of sensors 22 to infer the composition, homogeneity, color, temperature, and clinically correlated diagnostics about the fluids and be used to determine fluid volume. This data can be acquired on a continuous or intermittent basis.

In one embodiment, images 38 or video in different parts of the visual spectrum may also be used to determine volume. Analysis of fiducial markings either inside of the fluid housing or outside may be obscured to different degrees by the liquid (whether in visual spectrum, IR, etc.), and by analyzing these fiducial markers the information is used to infer fluid height and therefore volume. Additionally, an image sensor 22 looking directly downwards on a rising level of fluid 18 will see a slowly enlarging top-level surface area in the shape of an axial slice of the fluid. Due to the perspective of the image sensor 22, distant or low fluid 18 level will register as a smaller surface area and a closer or high fluid level will register as a high volume, which is used to derive exact volumetric measures.

In one embodiment, inference of these attributes can be achieved in many different ways. By creating a system 10 that stores the results of analyses as well as the outputs of many sensors 22 communicating with fluids 18 generated by patients 20, statistical methods for machine learning are applied to train programs running on the server to automatically predict the results of standard fluid analyses (e.g. chromatographic urine analysis) by recognizing patterns in data generated by the sensors. For instance, a trained program on the computer-controller 24 is able to automatically recognize the presence of urinary infection and even various specific infecting pathogens through the integration of information from the set of sensors 22, image sensors, volume sensors, as well as patient 20 entered data, thereby obviating the need for direct sample testing in the future. Similar pattern recognition in the observation of nasogastric fluids leads a trained machine, e.g. system 10, to recognize the presence of a bowel obstruction without the need for radiographic intervention, or proposes a ranked list of differential diagnoses.

In one embodiment, when replacing fluid housings 12, the computer-controller 24 can be used to save various metrics about the contents of the previous fluid housing, and integrate them with metrics of the new housing. This is achieved in the case of volumetric data by the computer-controller 24, which stores most recent volumetric measurements taken from an old fluid housing 12 and adds them to measurements from the new fluid housing to provide an accurate representation of cumulative volumes in a given period of time. This of course is not limited to volume, and may include additional fluid characteristics, such that those measurements may be represented (graphically, pictorially, or numerically) to represent instantaneous measurements as well as aggregate trends over defined periods of time.

In one embodiment, the system 10 includes fluid housings 12 that incorporate a port 40 for collecting samples of fluid 18 for human or machine-analysis either by manually withdrawing these samples through the port or by automating their collection, e.g. by on-board suction capabilities of the system. These samples can then be analyzed by the computer-controller 24 accessories or on-board machines. The sample collection and analysis can be programmed by the controller 24, either on demand, by scheduled order, or by prescribed protocol.

In one embodiment, the fluid housings 12 can be connected to waste-management systems to drain the contents 18 of the fluid housings. The drainage of the fluid housings' 12 contents 18 can be programmed by the controller 24 either on demand, by scheduled order, or by prescribed protocol.

In one embodiment, records, images 38 or videos of patient 20 outputs 18 being collected so that humans 28 can interpret them and make clinical decisions remotely. System 10 includes computer analysis and machine learning to both standardize interpretation of imaging characteristics (including but not limited to color, consistency, homogeneity, particulates, etc.) based upon algorithms for their analysis and signal processing. System 10 includes using machine-learning techniques, signals from a camera sensor 22 that are interpreted to glean data previously limited to microscopy—presence of abnormal cells, molecules, infection, etc., and it can be used to predict the presence of statistically likely pathogens or diagnoses.

In one embodiment, system 10 includes fluid-receptacle 12 that interfaces directly with tubes 42 leading from various points of outflow of patient fluids 18. These include a urinary catheter dwelling within the bladder and conveying by gravity urine outwards through a tube; a nasogastric suction tube utilizing built-in suction to generate outflow of gastric contents; the surgical-site drain which also uses negative-pressure to generate the outflow of serosanguinous fluids collecting at operated sites; and rectal tubes through which gravity guides the outflow of colonic fluids. All of these tubes 42, as well as other components, connect to a lid-like tube-adapter at the top of the fluid housing 12.

In one embodiment, the end of each of these tubes 42 interfaces with preexisting catheters and drains 16 that divert these fluids 18 from the patient's 20 body through the tubes. System 10 includes a set of adapters on multi-y-connectors at the end of each fluid-diverting tube 42, which can be used to fit either the fluid-diverting bodily drain 16 itself, or to the receptacle 12 to which it is connected at manufacture.

In one embodiment, system 10 uses both gravity and negative pressure generated both internally and externally to galvanize the movement of fluids 18 from the tubes 42 to the fluid housing 12. The fluid housing 12 itself, like the tubes, are disposable, and it externally fits into a molded bracketing system, which has a sturdy base with wheels to facilitate portability. A capacitive sensor 22 affixed to system and interfacing with the inserted fluid housing 12 determines the volume of fluid 18 content stored therein. This sensor 22 does not directly touch any fluids 18 but measures their level within the fluid housing 12. The level is recorded by the computer-controller 24 which infers a fluid volume from the registered fluid level, structures this data, and allows it to be either sent to or called from an electronic health record system at exact, programmable time intervals. In alternative embodiment, the level is recorded by computer-controller 24 that sends it via a Wifi-enabled, Bluetooth-enabled, cellular-data-enabled, or wired transceiver to a remote server. This server then process this data and infers a fluid volume from the registered fluid level, structures this data, and allows it to be either sent to or called from an electronic health record system at exact, programmable time intervals.

In one embodiment, system 10 also contains a camera system which can capture images 38 of the fluid 18 contained within the fluid housing 12. Either through programmable schedules or on demand, clinicians or other authorized users 28 of this clinical data can access both volumetric data—presented in numerous ways through associated software—and photographic data through any mobile or non-mobile connected device, including a telephone, smart-watch, tablet computer, laptop computer, or desktop computer. System 10 uses statistical methods to analyze the photographic 38 data, providing insights to clinicians 28 about the homogeneity of color and texture (via methods assessing dispersion), as well as average color and how it might correlate with various pathologies.

IN one embodiment, the system 10 provides a warning remote, that can allow a patient 20 to record when he is voiding or passing feces 18 by pressing a button, to allow for the recording of frequency. In an alternate embodiment, this button is packaged in a detachable format and affixed in the patient's 20 room—either near the bathroom or near the urinal to facilitate easy connection. In another embodiment, described as “patient-controlled charting,” system 10 includes a downloadable app for patients 20 to describe their fluids-related 18 experience, both quantitatively and qualitatively on their personal computing device, which can transmit this data to the computer-controller 24.

In one embodiment, system 10 uses as a touch-enabled LCD screen to facilitate device configuration, a barcode reader, and the Luer lock, to connect tubing, as well as a sampling system. The sampling system consists of a Luer-lock-enabled access-point on the fluid housing 12 and an automatic, programmable system for connecting to it sampling fluid housings (they can also be connected manually). In another embodiment, system 10 includes an on-board negative pressure vacuum system 16 which interfaces with select fluid housing 12 through an opening, as well as an elastic or spring-based system which keeps fluid-drawing tubes organized and unlikely to be tangled or tripped-on, but also with enough slack to allow for sudden patient 20 movements. The vacuum system 16 consists of a vacuum pump, pressure regulator, manifold, one-way check valves (to prevent backflow), and a pressure relief valve (in case of a system malfunction).

In one embodiment, system 10 is connected via two-way communication with the database that carries profile 26, computer-controller 24, and the interface 34. This technology can be used during surgery, or in the outpatient, home setting for the collection of any fluid 18 leaving the patient. The usefulness of this can also be in time-saving of weighing a patient 20 for strict in's and out's in an ICU, as the weighing of a patient can be very time-intensive.

A working example of the system 10 in use is as follows. In one embodiment, a new patient 20 arrives to a room with an indwelling urinary catheter, an indwelling NG-tube, a closed-suction surgical drain, and a rectal tube.

The patient's nurse 28 rolls in system 10, which has a sturdy base with wheels to facilitate portability. S/he 28 opens four new fluid housings 12, which have tubing 42 pre-attached and which are color-coded to remind him/her which fluids 18 they drain. One at a time, the nurse 28 attaches the correct connector to each fluid output: the urinary catheter bag, the Rectal tube bag, and the NG tube, which is currently disconnected from suction.

Each of these old-fashioned drains come with tubing 42 which can be reconnected to include a bypassing y-connector, allowing for diversion of fluids to the system 10. The nurses 28 uses this system to divert flow to the system 10. In the case that a y-connector is not available, s/he 28 also uses a special, multiperforated connector, which slides into any irregular suction surgical drain bulb and creates a tight seal where it enters with a slide-on cuff that can be tightened (this connection is used as an alternative when a y-connector cannot be inter-placed between tubing and suction drain).

Once these tubes 42 are connected, the nurse 28 uses either his/her mobile device or the digital visual display attached to system 10 in conjunction with a barcode reader to (1) scan the patient's 20 identifying wrist band or enter these details manually, (2) use a drop-down menu within the user interface to pair a given tube with a given fluid-label, and (3) to program measurements, pictures, and sampling as ordered by the relevant physician.

Once these are connected and operating, certain selections which require negative-pressure for fluid 18 collection will be automatically identified by the associated software of system 10, which will then begin administering the pre-programmed negative pressures to those fluid housings 12. The relevant physician 28 will then have software on his/her computing device which, given appropriate HIPAA and credential screening, will allow him/her remote access to repeat measures, pictures, and order-changes for system 10 in a given patient's 20 room within his/her service or hospital location.

Fluids 18 will collect, and system 10 software component will allow physicians 28 and nurses to view the latest observations remotely of the patient 20. Clinicians 28 can set pre-determined threshold values to trigger alarms (sent to local system 10 screens as well as push-notifying mobile app users) corresponding to adequate, inadequate, or greater than expected outflows of fluid.

Once fluids 18 collect to a pre-determined level, the nurse 28 will receive a warning that the appropriate fluid housing 12 should be changed. S/he 28 will go to the patient's 20 room, disconnect the relevant fluid housing 12 and detach it from the patient's 20 drain 16 or drain bag. S/he 28 will then repeat the original step of opening a new fluid housing 12 and attaching it to system 10 and to the patient 20. Fluid 18 collection will continue as before, based on the original programming instructions.

If the nurse 28 should need to flush any of these drains 16 with clean fluid (commonly performed to facilitate flow, clear obstructing debris, or provide space for sump-suction at a drain's end), s/he can use the system 10 software to document the amount flushed so as to not bias total measures.

If the patient 20 would like to stand up and ambulate, s/he can do so readily because system 10 is portable on wheels. If the patient 20 would like to document such movement or a sip of water, or emesis, urination, or bowel-movement, the patient can accomplish this by system 10 pre-manufactured buttons, the system 10 patient remote, or the system 10 patient-facing app that s/he can download to his/her computing device. Moreover, this app can be programmed to send prompts at random or scheduled time intervals to interrogate patients 20 regarding basic clinical rounds questions: review of systems (i.e. how the patient feels for each organ system), bowel and bladder habits/changes, emesis, etc. This interaction is called “Patient-Controlled Charting,” and is any information directly entered into an Electronic Medical Record, e.g. profile 26, by the patient described therein. This “Patient-Controlled Charting” information can be combined along with the photographic data 38 and physician-set 28 parameters to provide useful clinical information or have predictive value about a patient's condition or course. In one embodiment, the Patient-controlled-charting includes physical buttons, labeled “Ate,” “Drank,” “Urinated,” “Bowel Movement,” and “Vomited,” and are placed around patient 20 room and/or carried by system 10.

If the physician 28 would like to observe changes, s/he can use the system 10 software on his/her computing device to do so or make changes to observation scheduling. The background of the software will also interface with the hospital's electronic medical record, either through an API or through HIPAA protected text messages or through other relevant apps. In one embodiment, this communication consist of a URL hyperlink/button which the EMR-operating physician 28 can click to quickly be transferred to the relevant record 26 of observations. It may also take the form of information embedded directly into the EMR.

In one embodiment, the sensor 22 is placed in various places along the tubing 42 or fluid housing 12, based on its internal technology. In another embodiment, sensor 22 is capacitive and communicates with the computer-controller 24.

In one embodiment, the system 10 includes a connector that incorporates a multi-porous appendage that can be inserted into irregular closed-suction or passive-suction devices to suction-out their contents. Such connector includes a cuff portion to secure this connection.

System 10 includes a “Setup and Status” portion of profile 26, used to initialize the system for a new or returning patient 20. Displays basic operating status, including instantaneous volumetric data. Displayed on built-in LCD screen, but also available through mobile or desktop devices. When used by mobile devices, uses a barcode scanner through mobile device's camera to scan patients' wristbands and associate a given system 10 with a given patient 20. Not available to patients.

In one embodiment, system 10 includes a “Clinical” portion of profile 26, which provides visualizations of data collected by volumetric, photographic, and sampling equipment on multiple systems. Allows for basic analysis and exporting of de-identified data to CSV or other text formats. Not available to patients.

In one embodiment, system 10 includes a “Patient View” portion of profile 26, which provides patients 20 an overview of their own drains 16 and brief educational material on how and why they work. Incorporates a virtual “patient-controlled charting” set of buttons for documenting when they eat, drink, use the restroom, or vomit. Can only interact with the system assigned to the patient-user.

In one embodiment, system 10 includes an “administrator” portion of profile 26, used by information technology workers 28 to calibrate aspects of the back-facing portion of the software.

In one embodiment, system 10 provides control i.e. whether the suction is constant or intermittent, the level of pressure the vacuum 16 can generate, and whether it will act like a wall suction device or more of a negative pressure closed suction device such as you would find with a JP bulb (air is removed from a deformable fluid housing which generates this negative pressure.)

In one embodiment, an Image-Sensor-based method for measuring attributes of patient fluids, including machine learning methods is used by the system 10. A patient's fluid outputs are collected in multiple fluid housing containers, as discussed previously. Multiple methods may be used to ascertain characteristics about the fluids collected within them using only an image sensor—e.g. a photographic camera, or a UV camera. These methods include multiple configurations of the image sensor(s) directed at the fluid housing(s) or within the fluid housings, and a computer program to direct the capture of an image of the fluid and to process it. Finally a computer program is used to identify the fluids collected within the housing units and to evaluate various attributes thereof. These include the volume, temperature, pH, presence of microbes (e.g. fungus, bacteria), concentration of cells and molecules (including squamous cells, red blood cells, white blood cells, nitrites, leukocyte esterase, glucose, bile). The following list describes the examplary configurations of fluid housing and tubing, fluid, image sensor, and computer program for image processing and attribute measurement.

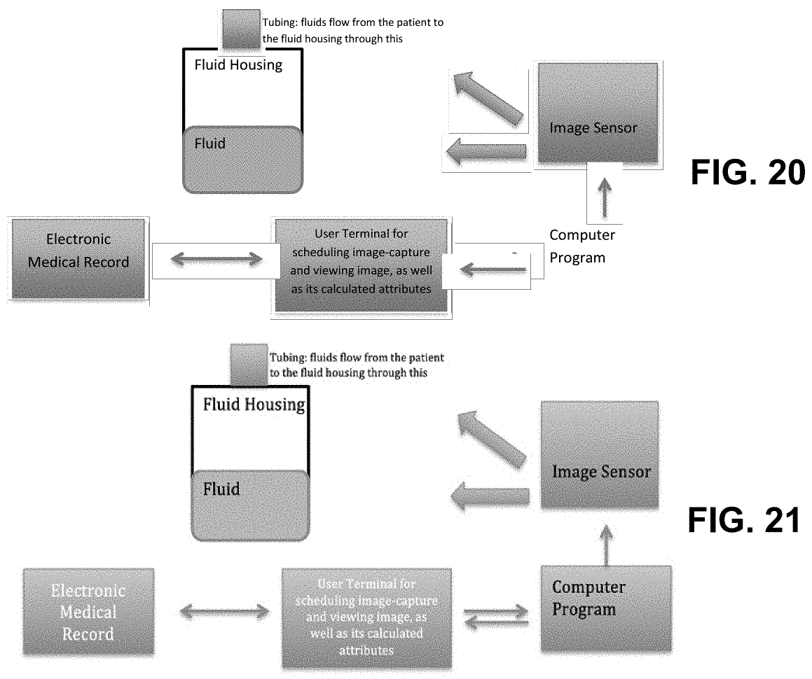

With additional reference to FIG. 20, in a first embodiment, the image sensor is positioned external to, but aimed at, the fluid housing, with lighting provided by either lighting in the facility (i.e. the patient's room), a back light from within or from behind the fluid housing, or as a “flash” lighting attached to the image sensor and also aimed at the fluid housing. The computer program directs it to capture an image. This is accomplished by a clinician pre-programming a schedule for image-capture (e.g. every 30 minutes for 48 hours), or on-demand when the clinician chooses to capture an image. The clinician operates these features through a terminal (discussed elsewhere).

In this first configuration, there are multiple methods of ascertaining attributes of interest such as exemplary embodiments labeled A-C below.

In method A, there are volumetric markings on the side of the fluid housing, facing the image sensor. These are visible in the image that was captured by the sensor and directed onwards to the user terminal or to the electronic medical record. The user ascertains the volume by looking at the image and can record the noted volume in the electronic medical record.

In a corollary method B, the computer program runs an algorithm, which automatically identifies the fluid housing, volumetric markings on the side of the fluid housing, and the parameters of the fluid within. The computer program uses these to return its “best-estimates” for correctly identifying the aforementioned attributes of the fluid. In this way, a well trained neural network in conjunction with an imaging sensor could get estimate or classify these attributes without using a volumetric sensor or human annotation.

To accomplish these tasks, the computer program utilizes an image classifier algorithm, based on a convolutional neural network (CNN), which has been described extensively in the literature and applied in industry In this approach an image is captured by the image sensor and passed to the computer program in the form of a matrix of pixels, each of a different color (this matrix can be described entirely by its height, in pixels, its width, n pixels, and a distribution of Red/Green/Blue color-model coordinates). The computer program then processes this multi-dimensional matrix through a convolutional neural network, consisting of mathematical convolution with a feature-matrix of weights, the result of which undergoes several steps to compress the information in the convolved matrix (including a pooling function to focus on the most important features of the captured image, followed by an activation function to reduce or remove interfering features). The resultant vector is then normalized and converted to a probability vector corresponding to the probabilities that the captured image belongs to each of many possible, pre-programmed classes (referred to later in our discussion as a classified image library). Each time that a pre-classified image (classified by a human or by another reliable sensor) undergoes the steps of the CNN and is classified, the computer program can adjust the weights of the feature-matrices in the CNN to improve classification accuracy (this utilizes a method known as back-propagation to minimize a function characterizing the error in classification). By passing many—perhaps thousands or millions—of images in this way through a CNN, the computer program can “learn” to efficiently classify images or parts thereof using for example the YOLO algorithm.

In one embodiment with additional reference to FIG. 21, the system 10 uses these methods by applying, them in a novel fashion for building a pre-classified image library. The system 10 does so by pairing the initial configuration of devices with (1) a volumetric sensor (for instance, a capacitive sensor), as well as (2) descriptions, or “classifications,” of the fluid's attributes provided by the clinician (or terminal user) and by the electronic medical record.

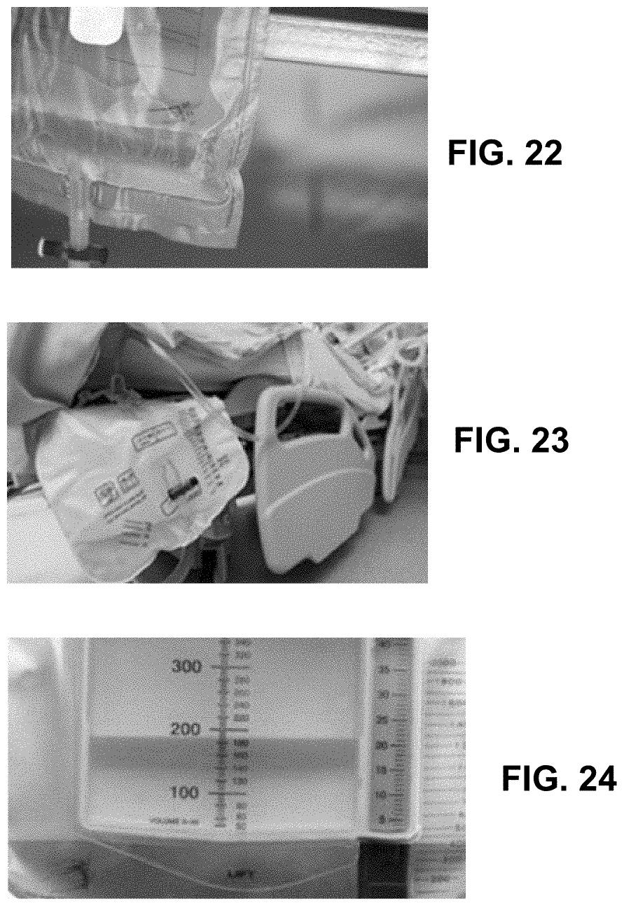

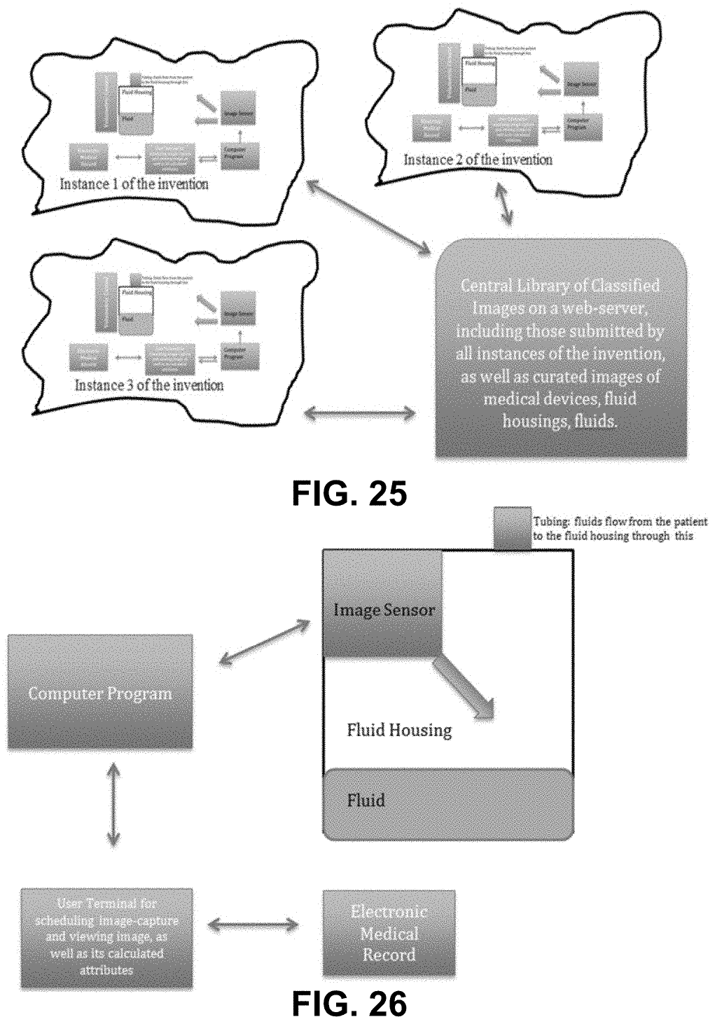

In one embodiment and with additional reference to FIGS. 22-24, each time that the image sensor of system 10 captures an image of the fluid housing, it is a priori classified according to the volumetric sensor and according to contemporary data from the EMR, which may have been entered by multiple other users. As such, the image sensor of system 10 may capture any of the following possible images such as FIGS. 22-24.

For example, at the time of image capture, FIG. 22 may be classified by an attached volumetric sensor, a user, and EMR data as the vector of labels: [Leg Bag Urine Meter, Urine, Clear, 150 mL, Urinalysis positive for glucose, leukocyte esterase, and white blood cells, Urinalysis negative for red blood cells, nitrates, patient temperature 97.8]. In similar fashion, FIG. 23 may be classified: [Bedside Urine Meter, Urine, Clear, 300 ml, Urinalysis negative for glucose, leukocyte esterase, white blood cells, negative for red blood cells, nitrites, patient temperature 98.4]. Similarly, FIG. 24 may be classified as [Bedside Urine Meter, Urine, Turbid Appearance, 190 mL, Urinalysis positive for glucose, leukocyte esterase, white blood cells, red blood cells, nitrites, patient temperature 100.4].

In one embodiment, each of these images would then be passed through a CNN within the computer program (or working in concert with it via a web-server). The CNN would classify each of these images, and be programmed to adjust itself to more accurately classify the image.

The system's 10 ability to classify such images accurately may be enhanced in at least 2 ways: (1) by “learning” to classify such images according to a novel, assembled and classified library of medical devices and fluid housings, and (2) by continuing to learn from each picture that it takes once deployed in the clinical setting. In this latter method, each instantiation of the system 10 would contribute classified images to a central library of classified images (working in concert with the computer program via web server), such that ail other instances of the system could “learn” from this central library.

In a corollary method C, the system 10 uses the same configuration as described above and may be augmented by the addition of fiduciary markers. These may include a bar-code or QR-code to mark volumetric measures on the side of the fluid housing or may be used to automate the linking of the given patient's electronic medical record with the computer program running on the current invention, which is to interact with the electronic medical record.

In another embodiment and with additional reference to FIG. 26, system 10 uses a different configuration that positions the image-sensor within the fluid housing, but otherwise performs very similar to configuration FIG. 20. For example, a method for determining attributes of the fluid requires volumetric markings on the side of the fluid housing and which are calibrated to be in the field of view of the image sensor. As before, working in concert with the computer program and with a remote web server, the image sensor can capture an image of the fluid within the fluid housing, and the user (or a clinician) can access it and label it from a user terminal.

In another embodiment as in the configuration of FIG. 20, a different method, the image sensor, as well as a library of images taken from within the fluid housing, may be used to “train” a CNN to accurately classify the attributes of the fluid within the housing. In this way, a well trained neural network in conjunction with a imaging sensor could get estimate or classify these attributes without using a volumetric sensor or human annotation.

In another embodiment as in the configuration of FIG. 20, a different method, the system 10 may be augmented to contain fiduciary markings, including a bar-code or QR-code to identify both volumetric measures, and patient-identifying measures.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Although this disclosure has been described in terms of certain implementations and generally associated methods, alterations, and permutations of these implementations and methods will be apparent to those skilled in the art. For example, the actions described herein can be performed in a different order than as described and still achieve the desirable results. As one example, the processes depicted in the accompanying figures do not necessarily require the particular order shown, or sequential order, to achieve the desired results. Systems and tools illustrated can similarly adopt alternate architectures, components, and modules to achieve similar results and functionality. For instance, in certain implementations, multitasking, parallel processing, and cloud-based solutions may be advantageous. Additionally, diverse user interface layouts, structures, architectures, and functionality can be supported. Other variations are within the scope of the following claims.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. A computer storage medium can be a non-transitory medium. Moreover, while a computer storage medium is not a propagated signal per se, a computer storage medium can be a source or destination of computer program instructions encoded in an artificially generated propagated signal. The computer storage medium can also be, or be included in, one or more separate physical components or media (e.g., multiple CDs, disks, or other storage devices), including a distributed software environment or cloud computing environment.

Networks, including core and access networks, including wireless access networks, can include one or more network elements. Network elements can encompass various types of routers, switches, gateways, bridges, load balancers, firewalls, servers, inline service nodes, proxies, processors, modules, or any other suitable device, component, element, or object operable to exchange information in a network environment. A network element may include appropriate processors, memory elements, hardware and/or software to support (or otherwise execute) the activities associated with using a processor for screen management functionalities, as outlined herein. Moreover, the network element may include any suitable components, modules, interfaces, or objects that facilitate the operations thereof. This may be inclusive of appropriate algorithms and communication protocols that allow for the effective exchange of data or information.

The operations described in this specification can be implemented as operations performed by a data processing apparatus on data stored on one or more computer-readable storage devices or received from other sources. The terms “computer-controller,” “data processing apparatus,” “processor,” “processing device,” and “computing device” can encompass all kinds of apparatus, devices, and machines for processing data, including by way of example a programmable processor, a computer, a system on a chip, or multiple ones, or combinations, of the foregoing. The apparatus can include general or special purpose logic circuitry, e.g., a central processing unit (CPU), a blade, an application specific integrated circuit (ASIC), or a field-programmable gate array (FPGA), among other suitable options. While some processors and computing devices have been described and/or illustrated as a single processor, multiple processors may be used according to the particular needs of the associated server. References to a single processor are meant to include multiple processors where applicable. Generally, the processor executes instructions and manipulates data to perform certain operations. An apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, module, (software) tools, (software) engines, or code) can be written in any form of programming language, including compiled or interpreted languages, declarative or procedural languages, and it can be deployed in any form, including as a standalone program or as a module, component, subroutine, object, or other unit suitable for use in a computing environment. For instance, a computer program may include computer-readable instructions, firmware, wired or programmed hardware, or any combination thereof on a tangible medium operable when executed to perform at least the processes and operations described herein. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network 25.

Programs can be implemented as individual modules that implement the various features and functionality through various objects, methods, or other processes, or may instead include a number of sub-modules, third party services, components, libraries, and such, as appropriate. Conversely, the features and functionality of various components can be combined into single components as appropriate. In certain cases, programs and software systems may be implemented as a composite hosted application. For example, portions of the composite application may be implemented as Enterprise Java Beans (EJBs) or design-time components may have the ability to generate run-time implementations into different platforms, such as J2EE (Java 2 Platform, Enterprise Edition), ABAP (Advanced Business Application Programming) objects, or Microsoft's .NET, among others. Additionally, applications may represent web-based applications accessed and executed via a network (e.g., through the Internet). Further, one or more processes associated with a particular hosted application or service may be stored, referenced, or executed remotely. For example, a portion of a particular hosted application or service may be a web service associated with the application that is remotely called, while another portion of the hosted application may be an interface object or agent bundled for processing at a remote client. Moreover, any or all of the hosted applications and software service may be a child or sub-module of another software module or enterprise application (not illustrated) without departing from the scope of this disclosure. Still further, portions of a hosted application can be executed by a user working directly at a server hosting the application, as well as remotely at a client.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), tablet computer, a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device, including remote devices, which are used by the user.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network 25. Examples of communication networks 25 include any internal or external network, networks, sub-network, or combination thereof operable to facilitate communications between various computing components in a system. A network may communicate, for example, Internet Protocol (IP) packets, Frame Relay frames, Asynchronous Transfer Mode (ATM) cells, voice, video, data, and other suitable information between network addresses. The network may also include one or more local area networks (LANs), radio access networks (RANs), metropolitan area networks (MANs), wide area networks (WANs), all or a portion of the Internet, peer-to-peer networks (e.g., ad hoc peer-to-peer networks), and/or any other communication system or systems at one or more locations.

The computing system can include clients and servers. A client and server are generally remote from each other and typically interact through a communication network 25. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

What is claimed is:

1. A system comprising:
a first-fluid-housing;
a drain in communication with the first-fluid-housing that pulls a first non-serosanguinous fluid from a patient into the first-fluid-housing;
a sensor that measures the first non-serosanguinous fluid entering the first-fluid-housing; and
a computer-controller in communication with the sensor and drain, the computer-controller includes a profile that identifies the patient, the first-fluid-housing, and records the sensor's first non-serosanguinous fluid measurements that any user can retrieve based upon permissions granted to each user.

2. The system of claim 1 further comprising a second-fluid-housing in communication with the drain that pulls a second-fluid generated by the patient into the second-fluid-housing, and the profile identifies the patient, the second-fluid-housing, and records the sensor's second-fluid measurements that any user can retrieve based upon permissions granted to each user.

3. The system of claim 1 further comprising an interface connecting the computer-controller to any user's computer based upon permissions granted to each user.

4. The system of claim 1 wherein the sensor records in the profile images of the first non-serosanguinous fluid generated by the patient that any user can retrieve based upon permissions granted to each user so the user can interpret them and make clinical decisions remotely.

5. The system of claim 4 wherein the user is at least one of human and machine-analysis, and images' characteristics include at least one of color, consistency, homogeneity, particulates, flow, volume, consistency, weight, composition, temperature, and clinically correlated diagnostics.

6. The system of claim 1 wherein the sensor measures the first non-serosanguinous fluid entering the first-fluid-housing by at least one of continuously, intermittently, in aggregate after the first-fluid-housing is exchanged, by flowrate, and by timestamp.

7. The system of claim 1 wherein the users and the patient communicate with each other based upon their permissions, and this communication allows the patient to directly enter descriptions of their subjective clinical experience into the profile.

8. The system of claim 1 wherein the computer-controller includes customizable thresholds that are set by the user based upon their permissions and prevents inappropriate settings of at least one of the drain, sensor, and user permissions.

9. A system comprising:
a first-fluid-housing;
a drain in communication with the first-fluid-housing that pulls a first non-serosanguinous fluid from a patient into the first-fluid-housing;
a sensor that measures the first non-serosanguinous fluid entering the first-fluid-housing;
a computer-controller in communication with the sensor and drain, the computer-controller includes a profile that identifies the patient, the first-fluid-housing, and records the sensor's first non-serosanguinous fluid measurements that any user can retrieve based upon permissions granted to each user;
a second-fluid-housing in communication with the drain that pulls a second-fluid generated by the patient into the second-fluid-housing, and the profile identifies the patient, the second-fluid-housing, and records the sensor's second-fluid measurements that any user can retrieve based upon permissions granted to each user; and
an interface connecting the computer-controller to any user's computer based upon permissions granted to each user.

10. The system of claim 9 wherein the sensor records in the profile images of the first non-serosanguinous fluid generated by the patient that any user can retrieve based upon permissions granted to each user so the user can interpret them and make clinical decisions remotely.

11. The system of claim 10 wherein the user is at least one of human and machine-analysis, and images' characteristics include at least one of color, consistency, homogeneity, particulates, flow, volume, consistency, weight, composition, temperature, and clinically correlated diagnostics.

12. The system of claim 9 wherein the sensor measures the first non-serosanguinous fluid entering the first-fluid-housing by at least one of continuously, intermittently, in aggregate after the first-fluid-housing is exchanged, by flowrate, and by timestamp.

13. The system of claim 9 wherein the users and the patient communicate with each other based upon their permissions, and this communication allows the patient to directly enter descriptions of their subjective clinical experience into the profile.

14. A system comprising:
a first-fluid-housing;
a drain in communication with the first-fluid-housing that pulls a first non-serosanguinous fluid from a patient into the first-fluid-housing;
a sensor that measures the first non-serosanguinous fluid entering the first-fluid-housing;
a computer-controller in communication with the sensor and drain, the computer-controller includes a profile that identifies the patient, the first-fluid-housing, and records the sensor's first non-serosanguinous fluid measurements that any user can retrieve based upon permissions granted to each user, the sensor records in the profile images of the first non-serosanguinous fluid generated by the patient that any user can retrieve based upon permissions granted to each user so the user can interpret them and make clinical decisions remotely, and the users and the patient communicate with each other based upon their permissions, and this communication allows the patient to directly enter descriptions of their subjective clinical experience into the profile.

15. The system of claim 14 further comprising a second-fluid-housing in communication with the drain that pulls a second-fluid generated by the patient into the second-fluid-housing, and the profile identifies the patient, the second-fluid-housing, and records the sensor's second-fluid measurements that any user can retrieve based upon permissions granted to each user.
